(19) United States
(12) Patent Application Publication
(10) Pub. No.: US 2024/0277406 A1
(43) Pub. Date: Aug. 22, 2024

(54) INTERNAL BIPOLAR RADIOFREQUENCY PROBE

(71) Applicant: ALFREDO ERNESTO HOYOS ARIZA, MIAMI, FL (US)

(72) Inventor: ALFREDO ERNESTO HOYOS ARIZA, MIAMI, FL (US)

(21) Appl. No.: 18/652,006

(22) Filed: May 1, 2024

Related U.S. Application Data
(63) Continuation-in-part of application No. 18/221,251, filed on Jul. 12, 2023.
(60) Provisional application No. 63/433,666, filed on Dec. 19, 2022.

(51) Int. Cl. A61B 18/16; A61B 18/00

(52) U.S. Cl. CPC A61B 18/16; A61B 2018/00321; A61B 2018/00714; A61B 2018/165

(57) ABSTRACT

A medical device for skin tightening, facial contouring, and body contouring-namely, an internal bipolar radiofrequency probe. The medical device may include: a housing, a first pole structurally disposed inside the housing and proximal to a first end of the housing, a second pole structurally disposed inside the housing and at a first distance away from the first pole, an isolation plate, and at least one sensor. Additionally, the present invention relates to a medical device that does not cause significant downtime in patients; presents a lower likelihood of potential to burn the skin of a patient, as compared to traditional radiofrequency devices by treating individual treatment sectors on the patient through a series of rapid energy bursts; and is practical, thereby permitting use in a wide variety of situations and contextual circumstances.

### CLAIM OF PRIORITY

The present application is a Continuation-In-Part (CIP) patent application of and claims priority to a previously filed, U.S. Non-Provisional patent application, namely, that having Ser. No. 18/221,251 and a filing date of Jul. 12, 2023, and further, claims priority to a previously filed U.S. Provisional Application having Ser. No. 63/433,666 and filed on Dec. 19, 2022, both of which are incorporated by reference herein in their entireties.

### BACKGROUND OF THE INVENTION

### Field of the Invention

The present invention is directed to a medical device for skin tightening, facial contouring, and body contouring, namely, an internal bipolar radiofrequency probe.

### Description of the Related Art

Skin aging is one of, if not the most, common cosmetic problems. Skin aging is complex in nature, having been shown to be caused by both “intrinsic” aging—also known as chronological aging, which is caused by the passage of time—and “extrinsic” aging—also known as “photoaging,” which occurs because of environmental influences, such as ultraviolet (UV) radiation or smoking. Both forms of aging have a common pathophysiology in that the skin is impacted by stressors that cause, inter alia, DNA damage and a decline in elastin and collagen fiber levels.

Over the past few decades, cosmetic procedures—such as liposuction, breast augmentation, rhinoplasty, tummy tucks, and facelifts—have grown in popularity worldwide. In their infancies, these procedures were largely surgical in nature, with patients experiencing substantial “downtime,” or the expected time to return to one's normal lifestyle. As one would expect, however, modern trends have shown that patients prefer minimally invasive or non-invasive options, which have less downtime. Unfortunately, many modern minimally invasive or non-invasive cosmetic procedures, including the use of chemical peels and ablative lasers, are still associated with substantial downtime and a plethora of potential post-operative complications.

With the increasing interest in minimally invasive or non-invasive cosmetic procedures and anti-aging among the public, there has been a significant push to create new ways of combating the same cosmetic problems that have plagued humanity. One such advancement lies in the field of radiofrequency (RF) technology, which has recently emerged as a novel treatment option to address skin laxity of both the face and body (e.g., soft tissue contraction, subdermal adipose remodeling, and rejuvenation of the face and body) while simultaneously providing potential benefits such as substantially reduced downtime and fewer post-operative complications (e.g., fewer post-operative pigmentation changes than laser treatments). RF technology is frequently used by medical professionals in conjunction with surgical procedures—such as liposuction—to achieve both tissue retraction and improved contouring of a patient's face and/or body, but May also be used on patients who are do not wish to undertake or are not candidates for a more invasive procedure.

RF technology employs the use of electromagnetic (EM) signals, namely RF energy, which is the term given to high-frequency alternating electrical current at the frequency range traditionally used for radio-wave communication. Medical devices that emit RF energy produce a change in the electrical charges of any treated tissue(s)—creating an electron movement—and the resistance of the treated tissue(s) to the electron movement generates heat (i.e., an electrothermal reaction in the treated tissue(s)). Tissues with a higher impedance, such as subcutaneous fat, are the traditional targets of RF technology, as these tissues generate more heat than others. By delivering RF energy—to the dermis and specifically not to the epidermis—which is ultimately converted into thermal energy upon resistance by any treated tissue(s), RF technology achieves both skin tightening and adipocyte apoptosis. Therefore, RF technology offers a method for non-ablative skin rejuvenation that can be applied to all kinds of skin, which is particularly useful for skin tightening and wrinkle reduction in the face and body.

Within the realm of RF technology, two of the most common embodiments are monopolar RF devices (a “MRFD”) and bipolar RF devices (a “BRFD”). The biggest difference between the two, however, is the difference in configuration. MRFDs function by delivering a current through one active electrode (or “pole”), which transmits the current to a grounding electrode (e.g., a grounding pad) at a specified distance away from the sole active electrode. BRFDs, on the other hand, traditionally utilize two active electrodes—wherein at least one of which contacts the skin—and have the current flow both between the two electrodes and to regionally targeted tissue(s).

As a result of the respective configurations, MRFDs can achieve high current penetration depth, while BRFDs can achieve a penetration depth of about half the distance between the two active electrodes (i.e., the energy distribution is more localized and controlled as compared to MRFDs)—as the current flows through the shortest path between the electrodes, thereby limiting the depth of the thermal response induced by the RF energy. While this may seem like MRFDs are preferable, high penetration of an emitted current in this context is associated with significant pain levels in patients. Accordingly, as compared to MRFDs, BRFDs deliver less discomfort and pain.

Furthermore, MRFDs, unlike BRFDs, cause volumetric and uniform heating of any impacted tissue(s), thereby requiring, in many instances, active cooling of the skin to reduce the likelihood of epidermal injury. Moreover, in comparison to BRFDs, patients who undergo procedures using MRFDs are associated with an increased likelihood of burns, scarring, ocular irritation, and corneal epithelial erosion. As such, BRFDs tend to be preferable over MRFDs, at least for the reasons described above.

Furthermore, and as alluded to above, BRFDs traditionally utilize two active electrodes and have the current flow both between them and to regionally targeted tissue(s). Usually included in the two active electrodes are an internal pole—which refers to the electrode inserted into or placed beneath the skin in proximity to the treatment area—and an external pole—which refers to the electrode placed on the surface of the skin, opposite the internal pole. The configuration of the internal and external poles completes the circuit of RF energy, allowing the internal pole to deliver the RF energy to the regionally targeted tissue(s) and allowing the current to pass through the regionally targeted tissue(s) to the external pole—thereby allowing for the controlled flow of current between the internal and external poles. However, although this classic form of BRFD presents a lower likelihood of, inter alia, burns (as compared to MRFDs)—and thus is seemingly much more preferable to MRFDs—the combination of an internal pole and an external pole still poses a relatively high risk of burns because the specific actions undertaken by the BRFDs necessarily occur between the dermis, epidermis, and fatty tissue(s).

Moreover, traditional BRFDs are not as effective for uses in certain areas of the body (e.g., areas with accentuated curvatures, hard-to-reach areas with deep targeted tissues). In fact, using traditional BRFDs in such cases is a largely cumbersome task and does not easily reach the aforementioned hard-to-reach areas, and even if able to, necessarily cannot operate with a high degree of accuracy. Therefore, because traditional BRFDs generally have their external poles located on human skin, the ability to access certain areas is limited in terms of physical ability and in terms of accuracy.

As such, a device is therefore required that provides for an alternative to the common current forms of radiofrequency technology with respect to skin tightening, facial contouring, and body contouring. Specifically, there is a need in the art for a medical device employing bipolar RF technology that is (1) safe for use on human patients—thereby not posing any significantly worse health concerns than methods currently offered—(2) is at least equally as effective as traditional BRFDs in addressing skin laxity of the face and body and/or tissue retraction; (3) is non-invasive or minimally invasive, thereby increasing the number of patients who are potential candidates for such treatment; and (4) does not cause significant downtime in patients. Additionally, there is a need in the art for such a device that (5) has a lower likelihood of potential to burn the skin of a patient and (6) allows for use in certain areas of the body (e.g., areas with accentuated curvatures, hard-to-reach areas with deep targeted tissues), thereby allowing any respective medical personnel to achieve a more precise and targeted delivery to desired tissue(s) (e.g., subcutaneous cellular tissue, superficial fascial system, reticular dermis, etc.). It is further desired that the present invention be practical—thereby permitting use in a wide variety of situations and contextual circumstances—and inexpensive-thus allowing for widespread use in several medicinal disciplines.

### SUMMARY OF THE INVENTION

In view of the disadvantages that come with using the aforementioned radiofrequency devices, the present invention is directed to a medical device for skin tightening, facial contouring, and body contouring which provides a practical device that is safe for use on human patients; is at least equally as effective as traditional radiofrequency devices in addressing skin laxity of the face and body and/or tissue retraction; is non-invasive or minimally invasive; does not cause significant downtime in patients; has a lower likelihood of potential to burn the skin of a patient; and allows for use in certain areas of the body (e.g., areas with accentuated curvatures, hard-to-reach areas with deep targeted tissues). As used herein, the term “between about” refers to the tolerance of values within the standard of error to a person of ordinary skill in the art.

As may be understood with reference to the term “undesired locations,” such locations May refer to areas or structures within the human body where the current should not flow or reach during the operation of the medical device. By way of non-limiting example, these “undesired locations” may include (1) surrounding healthy tissues, namely, tissue(s) beyond the treatment area; and (2) critical structures, namely, organs or other structural components.

As may be understood with reference to the term “therapeutic temperature,” such a temperature refers to a range of temperatures at which the medical device of the present invention may effectively perform its desired skin tightening, facial contouring, and body contouring functions. By way of non-limiting example, the “therapeutic range” of the present invention in an embodiment that provides for continuous operation may comprise the range of between about 38° C. to 42° C. Alternatively, for an embodiment described herein wherein fractional bursts are emitted, a preferred “therapeutic range” of each burst will be between 85° C. to 90° C., especially in an embodiment that provides for multiple bursts per second.

Furthermore, and as may be understood with reference to the term “cut-off temperature,” such a temperature refers to the temperature limit or threshold that, if exceeded by the medical device of the present invention operating in continuous mode, will likely cause harm to a patient or to any tissue(s) surrounding a target tissue(s). By way of non-limiting example, the “cut-off temperature” of the present invention operating in continuous mode may be between about 68° C. to 70° C. Conversely, when operating in burst mode the “cut-off temperature” would be much higher and typically will not be as applicable as the number of bursts per second and/or per square centimeter.

In more specific terms, the medical device for skin tightening, facial contouring, and body contouring may comprise a housing; a first pole, the first pole structurally disposed inside the housing and proximal to a first end of the housing; a second pole, the second pole structurally disposed inside the housing and at a first distance away from the first pole; and may be configured such that the first pole and the second pole are structurally disposed to allow for the transfer of radiofrequency energy between the first pole and the second pole. By way of non-limiting example, the housing may be comprised of at least one material chosen from a list of housing materials, the list of housing materials comprising ceramic and at least one metal. In at least some embodiments of the present invention, the first pole is connected to a device capable of producing RF energy. By way of additional non-limiting example, in at least some embodiments of the present invention wherein the first pole is connected to a device capable of producing RF energy, the first pole is disposed to transfer RF energy to the second pole. In at least some other embodiments of the present invention, the second pole is connected to a device capable of producing RF energy. By way of yet additional non-limiting example, in at least some embodiments of the present invention wherein the second pole is connected to a device capable of producing RF energy, the second pole is disposed to transfer RF energy to the first pole. In either of the aforementioned forms of the present invention, in such embodiments wherein either the first pole or the second pole is connected to a device capable of producing RF energy—and successive to an insertion of the medical device of the present invention onto or underneath (i.e., into) the skin of a patient—the device capable of producing RF energy is activated, thus generating RF energy and transferring it to either the first pole or the second pole (and thus successively to the second pole or first pole, respectfully). In doing so, the medical device of the present invention effectively creates an electrical circuit between the energy source and the respective poles. As a result, any tissue(s) exposed to the RF energy will resist the current, thereby generating resistive heating (i.e., the conversion of electrical energy into thermal energy). However, and as previously explained, the resultant thermal energy is essentially a controlled flow of current between the first and second poles.

Additionally, the present invention is directed to a medical device for skin tightening, facial contouring, and body contouring that may comprise a housing; a first pole, the first pole structurally disposed inside the housing and proximal to a first end of the housing; a second pole, the second pole structurally disposed inside the housing and at a first distance away from the first pole; the device configured such that the first pole and the second pole are structurally disposed to allow for the transfer of radiofrequency energy between the first pole and the second pole; and an isolation plate, the isolation plate structurally disposed between the first pole and the second pole. In such instances wherein the medical device of the present invention comprises an isolation plate, the isolation plate necessarily functions by serving as an electrical grounding point for any RF energy generated. In such cases, the isolation plate may provide a low-resistance path for the RF energy to flow, diverting the RF energy away from undesired locations (i.e., the isolation plate may provide a clear path for the RF energy to flow between the first pole and the second pole of the medical device of the present invention, thus ensuring that the RF energy is focused on a targeted area). By way of non-limiting example, the housing may be comprised of at least one material chosen from a list of housing materials, the list of housing materials comprising ceramic and at least one metal. In at least some embodiments of the present invention, the first pole is connected to a device capable of producing RF energy. By way of additional non-limiting example, in at least some embodiments of the present invention wherein the first pole is connected to a device capable of producing RF energy, the first pole is disposed to transfer RF energy to the second pole. In at least some other embodiments of the present invention, the second pole is connected to a device capable of producing RF energy. By way of yet additional non-limiting example, in at least some embodiments of the present invention wherein the second pole is connected to a device capable of producing RF energy, the second pole is disposed to transfer RF energy to the first pole. By way of yet additional non-limiting example, in at least some embodiments of the present invention comprising an isolation plate, the isolation plate is disposed to redirect RF energy from undesired locations.

Moreover, the present invention is directed to a medical device for skin tightening, facial contouring, and body contouring that may comprise a housing; a first pole, the first pole structurally disposed inside the housing and proximal to a first end of the housing; a second pole, the second pole structurally disposed inside the housing and at a first distance away from the first pole; the device configured such that the first pole and the second pole are structurally disposed to allow for the transfer of radiofrequency energy between the first pole and the second pole; an isolation plate, the isolation plate structurally disposed between the first pole and the second pole; and at least one sensor, the at least one sensor structurally configured to collect intraoperative data. By way of non-limiting example, the housing may be comprised of at least one material chosen from a list of housing materials, the list of housing materials comprising ceramic and at least one metal. In at least some embodiments of the present invention(s), the first pole is connected to a device capable of producing RF energy. By way of additional non-limiting example, in at least some embodiments of the present invention wherein the first pole is connected to a device capable of producing RF energy, the first pole is disposed to transfer RF energy to the second pole. In at least some other embodiments of the present invention, the second pole is connected to a device capable of producing RF energy. By way of yet additional non-limiting example, in at least some embodiments of the present invention wherein the second pole is connected to a device capable of producing RF energy, the second pole is disposed to transfer RF energy to the first pole. By way of yet additional non-limiting example, in at least some embodiments of the present invention comprising an isolation plate, the isolation plate is disposed to redirect RF energy from undesired locations.

With specific regard to embodiments of the present invention comprising at least one sensor, the at least one sensor comprising a thermostat, the thermostat disposed to measure at least one temperature of at least one tissue. In such embodiments wherein the at least one sensor comprises a thermostat, the thermostat is disposed to produce a first indication signal and a second indication signal. In such embodiments wherein the thermostat is disposed to produce a first indication signal and a second indication signal, the first indication signal may indicate a first temperature range and the second indication signal may indicate a second temperature range. By way of non-limiting example, in such embodiments wherein the first indication signal indicates a first temperature range and the second indication signal indicates a second temperature range, the first temperature range comprises at least one therapeutic temperature. By way of additional non-limiting example, in such embodiments wherein the first indication signal indicates a first temperature range and the second indication signal indicates a second temperature range, the second temperature range comprises a cut-off temperature. By way of yet additional non-limiting example, in such embodiments wherein the second temperature range comprises a cut-off temperature, the medical device may comprise a shut-down mechanism, the shut-down mechanism disposed to turn off the medical device to avoid injuring a patient via burn or other affliction.

With further specific regard to another embodiment of the present invention comprising at least one sensor, the at least one sensor comprising a burst controller structured to control the generation of fractional bursts within a desired therapeutic temperature range, including primarily the number of such bursts or pulses per second that are generated. Said at least one sensor May also comprise a position sensor structured to identify treatment sectors which have been treated in order to facilitate a systematic coverage of a treatment area without missing and/or re-treating a particular treatment sector.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic, front and side perspective view of at least one embodiment of the medical device for skin tightening, facial contouring, and body contouring.

FIG. 2 is a schematic, top perspective view of the exterior of at least one embodiment of the medical device for skin tightening, facial contouring, and body contouring.

FIG. 3 is a schematic, cross-sectional perspective view of at least one embodiment of the medical device for skin tightening, facial contouring, and body contouring.

Like reference numerals refer to like parts throughout the several views of the drawings.

### DETAILED DESCRIPTION OF THE PREFERRED EMBODIMENT

The invention now will be described more fully hereinafter with reference to the accompanying drawings in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

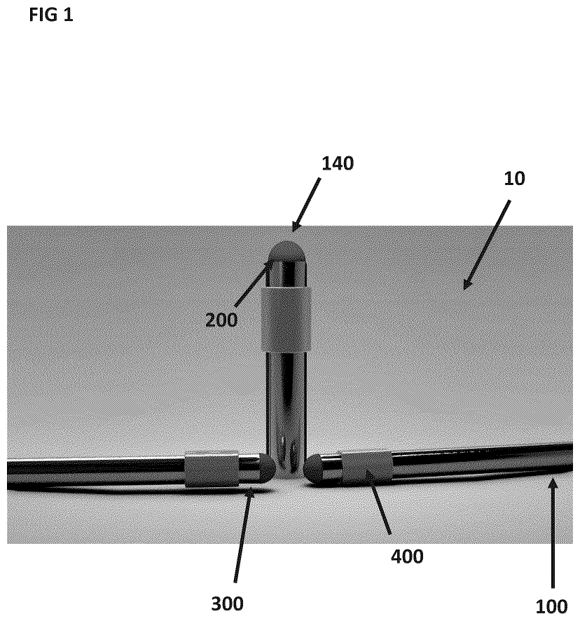

Turning now descriptively to the figures, FIGS. 1-3 illustrate an inventive medical device for skin tightening, facial contouring, and body contouring which is safe for use on human patients; is at least equally as effective as traditional radiofrequency devices in addressing skin laxity of the face and body and/or tissue retraction; is non-invasive or minimally invasive; does not cause significant downtime in patients; has a lower likelihood of potential to burn the skin of a patient; and allows for use in certain areas of the body (e.g., areas with accentuated curvatures, hard-to-reach areas with deep targeted tissues).

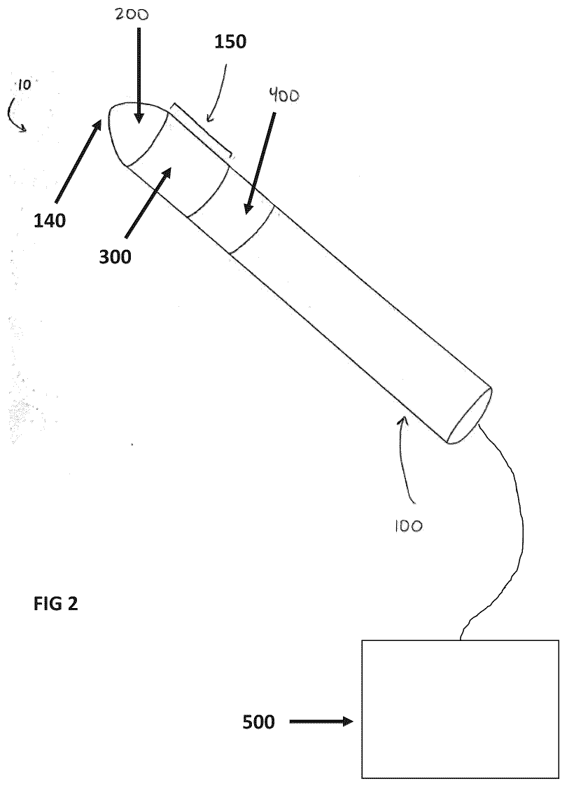

The medical device represented as 10 in FIGS. 1-2 may comprise a housing 100; a first pole 200, the first pole 200 structurally disposed inside the housing 100 and proximal to a first end 140 of the housing 100; a second pole 400, the second pole 400 structurally disposed inside the housing 100 and at a first distance 150 away from the first pole 200; and may be configured such that the first pole 200 and the second pole 400 are structurally disposed to allow for the transfer of radiofrequency energy between the first pole 200 and the second pole 400. By way of non-limiting example, the housing 100 may be comprised of at least one material chosen from a list of housing materials, the list of housing materials comprising ceramic and at least one metal. In at least some embodiments of the present invention, the first pole 200 is connected to a device capable of producing RF energy 500. By way of additional non-limiting example, in at least some embodiments of the present invention wherein the first pole 200 is connected to a device capable of producing RF energy 500, the first pole 200 is disposed to transfer RF energy to the second pole 400. In at least some other embodiments of the present invention, the second pole 400 is connected to a device capable of producing RF energy 500. By way of additional non-limiting example, in at least some embodiments of the present invention wherein the second pole 400 is connected to a device capable of producing RF energy 500, the second pole 400 is disposed to transfer RF energy to the first pole 200.

Additionally, the present invention, seen in FIGS. 1-2, is directed to a medical device 10 for skin tightening, facial contouring, and body contouring that may comprise a housing 100; a first pole 200, the first pole 200 structurally disposed inside the housing 100 and proximal to a first end 140 of the housing 100; a second pole 400, the second pole 400 structurally disposed inside the housing 100 and at a first distance away from the first pole 200; the device configured such that the first pole 200 and the second pole 400 are structurally disposed to allow for the transfer of radiofrequency energy between the first pole 200 and the second pole 400; and an isolation plate 300, the isolation plate 300 structurally disposed between the first pole 200 and the second pole 400. In at least some embodiments of the present invention comprising an isolation plate 300, the isolation plate 300 is disposed to redirect RF energy from undesired locations. In such instances wherein the medical device 10 of the present invention comprises an isolation plate 300, the isolation plate 300 necessarily functions by serving as an electrical grounding point for any RF energy generated.

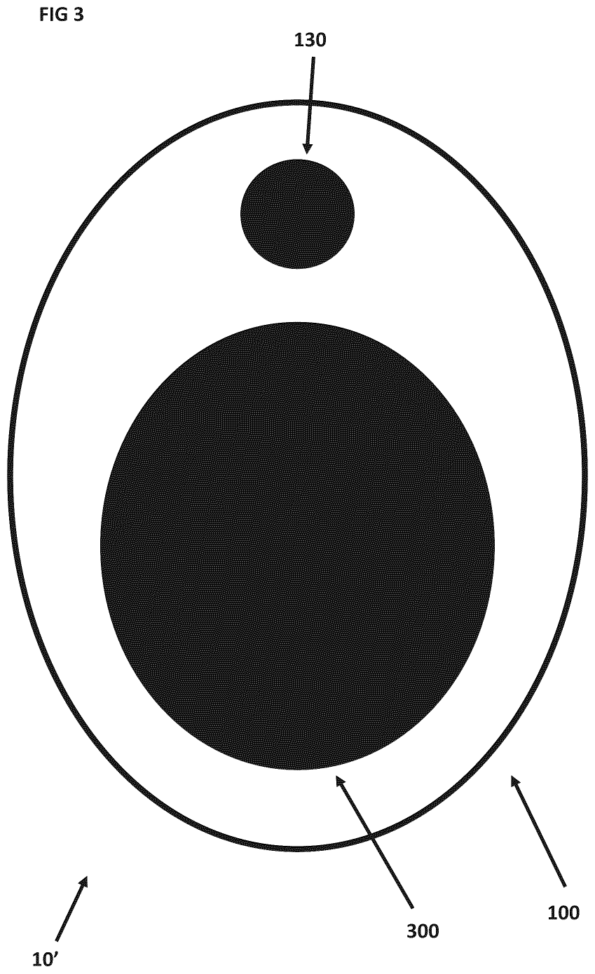

Moreover, and as can be seen in FIG. 3, the present invention is directed to a medical device 10′ for skin tightening, facial contouring, and body contouring that may comprise a housing 100; a first pole 200, the first pole 200 structurally disposed inside the housing 100 and proximal to a first end 140 of the housing, a second pole 400; the second pole 400 structurally disposed inside the housing 100 and at a first distance 150 away from the first pole 200; the device configured such that the first pole 200 and the second pole 400 are structurally disposed to allow for the transfer of radiofrequency energy between the first pole 200 and the second pole 400; an isolation plate 300, the isolation plate 300 structurally disposed between the first pole 200 and the second pole 400, and at least one sensor 130, the at least one sensor 130 structurally configured to collect intraoperative data. With specific regard to those embodiments of the present invention comprising at least one sensor 130, seen in FIG. 3, in one embodiment the at least one sensor 130 comprises a thermostat, the thermostat disposed to measure at least one temperature of at least one tissue. In such embodiments wherein the at least one sensor 130 comprises a thermostat, the thermostat is disposed to produce a first indication signal and a second indication signal—wherein the first indication signal may indicate a first temperature range and the second indication signal May indicate a second temperature range. By way of non-limiting example, in such embodiments wherein the first indication signal indicates a first temperature range and the second indication signal indicates a second temperature range, the first temperature range comprises at least one therapeutic temperature. By way of additional non-limiting example, in such embodiments wherein the first indication signal indicates a first temperature range and the second indication signal indicates a second temperature range, the second temperature range comprises a cut-off temperature. By way of yet additional non-limiting example, in such embodiments wherein the second temperature range comprises a cut-off temperature, the medical device may comprise a shut-down mechanism, the shut-down mechanism disposed to turn off the medical device to avoid injuring a patient via burn or other affliction.

Turning to another embodiment of the present invention, instead of providing continuous operation wherein a constant preferred therapeutic temperature is achieved, it may be preferred that the present invention have a fractional operation wherein systematic and controlled bursts of energy are produced. For example, rather than providing extended heating, which requires lower temperatures and a monitoring of the cut-off temperature, by operating in fractional, timed bursts that often last mere nanoseconds, a higher and more effective therapeutic temperature range can be achieved without risk of overheating the treatment area. Specifically, a burst operation mode provides for a very quick on and off pulsing which allows for both a rapid and effective temperature increase as well as a rapid cool down, thus minimizing negative impacts. In an optimal or preferred embodiment, a treatment area can be divided into individual treatment sectors, such as in a grid format. Further, it is preferred that each treatment sector have a defined area, such as for example one square centimeter in a preferred embodiment. In such an embodiment, the at least one sensor 130 can include a burst controller which controls how many bursts per second are generated and the temperature of each burst. Along these lines, in a preferred embodiment, for a standard one square centimeter treatment sector wherein there is a more superficial treatment area, the burst controller may direct one to three bursts per second, with each burst being a pulse in a therapeutic range of between 85° C. to 90° C. Of course, different treatment sites may require different burst frequencies and temperatures. By way of another non-limiting example, if a treatment area includes a denser or thicker area, such as near or in excess of 1.5 centimeters in thickness, it may be ideal to provide for a number of bursts (ex. 1-3) at a deeper location as well as one or more superficial bursts per second. This variable depth of the burst can be managed by multiple probes, by penetration of the probe for initial bursts and withdrawal for the superficial burst, or by having multiple contact points on the probe.

It is also understood that treatment areas will in many situations be larger than the preferred one square centimeter treatment sector. As such, it is understood that multiple burst treatments will often be necessary as all or multiple treatment sectors within a treatment area are subjected to bursts. Although a variety of techniques may be employed to ensure that an entire treatment area is impacted, in another embodiment, the at least one sensor may include a position sensor to modify, identify and track the various treatment sectors that have been treatment in order to ensure a systematic and desired coverage over the treatment area. Optimally, this will provide either a visual or audible indicator to direct an operator to treatment sectors that still require treatment and/or to treatment sectors that have already been fully treated. Yet another consideration that is achieved is to track which treatment sectors have been treated, but also to record how much time has based since that treatment, as it is contemplate that in some use cases multiple treatments at a specific treatment sector are desired, but spacing those treatments either too close or too far apart, both in time or location, may not be optimal.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A medical device for skin tightening, facial contouring, and body contouring comprising:
a housing,
a first pole, said first pole structurally disposed inside said housing and proximal to a first end of said housing,
a second pole, said second pole structurally disposed inside said housing and at a first distance away from said first pole, and
said first pole and said second pole structurally disposed to allow for the transfer of radiofrequency energy between said first pole and said second pole in timed bursts.

2. The medical device of claim 1, wherein said first pole is connected to a device capable of producing radiofrequency energy.

3. The medical device of claim 2, wherein said first pole is disposed to transfer radiofrequency energy to said second pole.

4. The medical device of claim 1, wherein said second pole is connected to a device capable of producing radiofrequency energy.

5. The medical device of claim 4, wherein said second pole is disposed to transfer radiofrequency energy to said first pole.

6. The medical device of claim 1 wherein said timed bursts comprise one to three pulses per second.

7. The medical device of claim 6 wherein said pulses operate in a temperature range of between 85° C. to 90° C.

8. A medical device for skin tightening, facial contouring, and body contouring comprising:
a housing,
a first pole, said first pole structurally disposed inside said housing and proximal to a first end of said housing,
a second pole, said second pole structurally disposed inside said housing and at a first distance away from said first pole,
said first pole and said second pole structurally disposed to allow for the transfer of radiofrequency energy between said first pole and said second pole in timed bursts, and
an isolation plate, said isolation plate structurally disposed between said first pole and said second pole.

9. The medical device of claim 8, wherein said first pole is connected to a device capable of producing radiofrequency energy.

10. The medical device of claim 9, wherein said first pole is disposed to transfer radiofrequency energy to said second pole.

11. The medical device of claim 8, wherein said second pole is connected to a device capable of producing radiofrequency energy.

12. The medical device of claim 11, wherein said second pole is disposed to transfer radiofrequency energy to said first pole.

13. The medical device of claim 8, wherein said isolation plate is disposed to redirect radiofrequency energy from undesired locations.

14. A medical device for skin tightening, facial contouring, and body contouring comprising:
a housing,
a first pole, said first pole structurally disposed inside said housing and proximal to a first end of said housing,
a second pole, said second pole structurally disposed inside said housing and at a first distance away from said first pole,
said first pole and said second pole structurally disposed to allow for the transfer of radiofrequency energy between said first pole and said second pole,
an isolation plate, said isolation plate structurally disposed between said first pole and said second pole, and
at least one sensor, said at least one sensor structurally configured to collect intraoperative data.

15. The medical device of claim 14, wherein said first pole is connected to a device capable of producing radiofrequency energy.

16. The medical device of claim 15, wherein said first pole is disposed to transfer radiofrequency energy to said second pole.

17. The medical device of claim 14, wherein said second pole is connected to a device capable of producing radiofrequency energy.

18. The medical device of claim 17, wherein said second pole is disposed to transfer radiofrequency energy to said first pole.

19. The medical device of claim 14, wherein said isolation plate is disposed to redirect radiofrequency energy from undesired locations.

20. The medical device of claim 14, wherein said at least one sensor comprises a burst controller structured to control the timing of energy bursts that result from the transfer of radiofrequency energy.

21. The medical device of claim 20, wherein said burst controller is further structured to set an optimal temperature of said energy bursts.

22. The medical device of claim 14 wherein the transfer of radiofrequency energy achieves therapeutic temperatures in the range of 85° C. to 90° C.

23. The medical device of claim 14, wherein the transfer of radiofrequency energy is emitted in bursts at a rate of at least two bursts per second.

24. The medical device of claim 14 wherein said at least one sensor comprises a position sensor structured to monitor and track treatment sectors that have been treated by the transfer of radiofrequency energy.

25. The medical device of claim 24 wherein said treatment sectors are approximately one square centimeter in size.
